(19) United States
(12) Patent Application Publication
(10) Pub. No.: US 2005/0106635 A1
(43) Pub. Date: May 19, 2005

(54) Compositions and methods for regulating thyroid hormone metabolism and cholesterol and lipid metabolism via the nuclear receptor car

(76) Inventors: Jodi Marie Maglich, Durham, NC (US); John Tomlin Moore, Durham, NC (US)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/506,647

(22) PCT Filed: Feb. 28, 2003
(86) PCT No.: PCT/US03/06089

Related U.S. Application Data
(60) Provisional application No. 60/361,906, filed on Mar. 4, 2002.

(51) Int. Cl. G01N 33/53; G01N 33/567; A61K 31/198

(52) U.S. Cl. 435/7.2; 514/567

(57) ABSTRACT

Methods for identifying agents which alter thyroid hormone metabolism or cholesterol and lipid metabolism by modulating expression and/or activity of the nuclear receptor CAR are provided. Also provided are compositions containing such agents and methods of using such agents to alter thyroid hormone metabolism or cholesterol and lipid metabolism in a subject. Administration of such agents is useful in the treatment of conditions such as obesity, cholesterolemia and dyslipidemia.

### FIELD OF THE INVENTION

The present invention relates to compositions and methods of using compositions comprising modulators for the nuclear receptor CAR to alter thyroid hormone metabolism, cholesterol and lipid metabolism and basal metabolic rate. As demonstrated herein, CAR ligands provide a means for selective modulation of thyroid hormone metabolism in tissues expressing this receptor. By modulating thyroid hormone metabolism in tissues such as the liver, the basal metabolic rate can be regulated as well as cholesterol and lipid metabolism. Compositions comprising a modulator for CAR can thus be used in the treatment of various conditions including, but not limited to, obesity, hypercholesterolemia, and dyslipidemia.

### BACKGROUND OF THE INVENTION

Thyroid hormone is a major regulator of energy homeostasis and metabolism. The levels of thyroid hormones are tightly controlled by several mechanisms, including feedback inhibition of thyroid hormone on the hypothalamic/pituitary/thyroid axis (Yen, P. M. Physiol. Rev. 2001 81:1097-1142), leptin secretion by adipose tissue (Iossa et al. Int. J. Obes. Relat. Metab. Disord. 2001 25(3):417-25), increased or decreased deiodination of T4 to T3 by peripheral tissues, and hepatic and renal metabolism of T4 and T3 to less active and more readily excreted metabolites (Larsen, P. R. and S. H. Ingbar (1992). The Thyroid Gland, in “Textbook of Endocrinology” Philadelphia, W. B. Saunders Co.). Thyroid hormone exerts its effects in virtually every tissue, causing a generalized increase in anabolism and catabolism. Ultimately, the effects of thyroid hormone are mediated by binding and activation of the nuclear receptors, TRα and TRβ. The most potent thyroid hormone receptor ligand is T3 and its regulation is key to maintaining overall caloric balance in the face of nutritional stress.

The liver plays a central role in thyroid hormone metabolism and multiple metabolic pathways have been elucidated (Wu, S.-Y. and T. J. Vissar, Eds. (1993). Thyroid Hormone Metabolism: Molecular Biology and Alternate Pathways. Boca Raton, Fla., CRC Press). In the liver, the majority of T4 to T3 conversion is catalyzed by type I deiodinase, which catalyzes outer ring deiodination of T4 to produce T3 as well as inner ring deiodination to produce reverse-T3 (rT3). Because of their effects on deiodinase activity, specific sulfotransferases play a key role in regulating this process. Sulfation of the phenolic hydroxyl of thyroid hormone substrates dramatically alters type I deiodinase enzyme activity. In studies utilizing rat type I deiodinase, T4 sulfation was found to decrease outer ring deiodination activity to undetectable levels, whereas inner ring deiodinase activity increased dramatically (Vₘₐₓ/K_{M} increased over 130-fold). Inner ring deiodinase activity results in the production of sulfated-rT3, a product with less activity on the thyroid hormone receptors and which is typically further conjugated and rapidly eliminated.

Other metabolic fates of thyroid hormone are also important in the liver. One example is conjugation of thyroid hormones by UDP-glucuronyltransferases, specifically by members of the UGT1 family. Glucuronidation increases the water solubility of its thyroid hormone conjugates and hence their biliary and urinary excretion (Burchell and Coughtrie Pharmacol. Ther. 1989 43:261-89). Additionally, thyroid hormones are processed by deamination, decarboxylation of side chains, conjugation with glutathione, and cleavage of the thyroid hormone ether linkage. One effect of these various pathways is to produce a complex array of thyroid hormone metabolites, many of which are active but whose detailed effects are poorly understood. Another effect of these various pathways is to reduce levels of T3. Considering the contribution of the liver to thyroid hormone metabolism, understanding the regulation of these various pathways is critical to a complete understanding of energy homeostasis.

Alterations in hepatic thyroid hormone levels also have an impact on cholesterol and lipid metabolism through modulation of gene expression in the liver (Feng et al. Mol. Endocrinol. 2000 14(7):947-955.). Increases in thyroid hormone results in a reduction in cholesterol levels and plasma triglycerides. This is in part due to an elevated clearance rate mediated by increased expression of hepatic low density lipoprotein receptors (Scarabottolo et al. Atherosclerosis 1986 59(3):329-333) and an increase in specific lipid-lowering liver enzymes such as cholesterol ester transfer protein (Berti et al. Metabolism 2001 50(5): 530-6), lipoprotein lipase, hepatic lipase (Valdemarsson et al. Acta Endocrinol. (Copenh) 1983 104(1):50-6; Ridgway, N. and Dolphin, P. J. Biochim. Biophys. Acta 1984 796(1):64-71; Ridgway, N. D. and Dolphin, P. J. J. Lipid Res. 1985 26(11):1300-13) and lecithin:cholesterol acetyl transferase (Berti et al. Metabolism 2001 50(5):530-6; Hulsmann et al. Biochem. Biophys. Res. Commun. 1977 79(3):784-8). Regulation of these genes is tightly controlled during various physiological challenges, such as during fasting. Because of the importance of these pathways in caloric balance, cholesterol, and lipid metabolism, modulating hepatic thyroid hormone metabolism could have pharmacological consequences.

The orphan nuclear receptor CAR, also referred to as NR1I3, is expressed mainly in liver, intestine, and kidney, the predominant sites of thyroid hormone metabolism. CAR activators such as 1,4-bis-[2,-(3,5-dichloropyridyloxy)] benzene (TCPOBOP) and phenobarbital, have been shown to affect T4 levels in rodents by unknown mechanisms. In rats, phenobarbital and high doses of TCPOBOP lead to an increase in glucuronidation of T4, resulting in decreased circulating levels of T4 (Barter, R. A. and Klaassen, C. D. Toxicol. Appl. Pharmacol. 1992 113:3642). This decrease is sufficient to enhance thyroid-stimulation hormone (TSH) levels (Kolaja, K. L. and Klaassen, C. D. Toxicol. Sci. 1998 46(1):31-7), thyroid cell proliferation, and to increase rates of carcinogenesis in rats co-treated with a tumor promoter (Diwan et al. Carcinogenesis 1996 17(1):37-43).

CAR has been proposed to function primarily as a xenobiotic sensor and various studies have been performed to characterize the xenobiotic sensing properties of CAR. In response to xenobiotics, CAR directly induces expression of the Cyp2b and Cyp3a genes, (Honkakosli et al. Mol. Cell. Biol. 1998 18(10):5652-8; Xie et al. Genes Dev. 2000 14(23):3014-23; Zelko et al. Biochem. Biophys. Res. Comm. 2000 277:1-6), as well as the gene encoding the glucuronidation enzyme UDP-glucuronyltransferase UGT1A1 (Sugatani et al. Hepatology 2001 33(5):1232-8). The products of these genes are generally involved in metabolism and clearance of xenobiotic substrates. Gene disruption experiments support this model, demonstrating that CAR knock-out mice are more sensitive to external agents (for example, the anesthetic zoxazolamine) compared to normal mice (Wei et al. Nature 2000 407(6806):920-3). Thus, the genes induced by CAR, the ligand activation spectrum of CAR, and genetic analyses support the role of CAR in xenobiotic protection.

WO 01/51045 discloses screening assays for identifying therapeutic agents for treating CAR-mediated disorders. In particular, use of CAR agonists to treat disorders such as hypercholesterolemia is suggested.

It has now been found that CAR is also involved in global energy homeostasis in response to fasting through modulation of thyroid hormone metabolism in the liver.

### SUMMARY OF THE INVENTION

An object of the present invention is to provide methods for identifying new therapeutic agents that alter thyroid hormone metabolism in extrathyroidal tissues and/or regulate basal metabolic rate and/or cholesterol and lipid metabolism via modulation of the expression and/or activity of CAR.

Another object of the present invention is to provide compositions comprising an agent the alters expression and/or activity of CAR that are useful in modulating thyroid hormone metabolism in extrathyroidal tissues and/or regulating basal metabolic rates as well as cholesterol and lipid metabolism. In a preferred embodiment, the agent is an antagonist of CAR expression and/or activity. Compositions of the present invention can be used in the treatment of conditions including, but not limited to, obesity, cholesterolemia, and dyslipidemia.

Another object of the present invention is to provide methods for modulating thyroid hormone metabolism in extrathyroidal tissues in a subject which comprise administering to the subject an agent which modulates activity and/or expression of CAR.

Examples of extrathyroidal tissues expressing CAR in which thyroid hormone metabolism can be modulated include, but are not limited to, liver, skeletal muscle, heart, brain, kidney and intestine.

Another object of the present invention is to provide methods for regulating basal metabolic rates or cholesterol or lipid metabolism in a subject which comprise administering to the subject an agent which modulates activity and/or expression of CAR. In a preferred embodiment, the agent is an antagonist of CAR expression and/or activity.

Yet another object of the present invention is to provide methods for treatment of a condition relating to altered basal metabolic rates or altered cholesterol or lipid metabolism in a subject which comprise administering to the subject an agent which modulates activity and/or expression of CAR. In a preferred embodiment, the agent is an antagonist of CAR expression and/or activity. Examples of conditions relating to altered basal metabolic rates or altered cholesterol or lipid metabolism include, but are not limited to, obesity, cholesterolemia, and dyslipidemia.

### DETAILED DESCRIPTION OF THE INVENTION

Thyroid hormone has potent effects on cholesterol, plasma lipid levels and on energy utilization Agents that modulate metabolism of this hormone are expected to be useful in the treatment of a variety of conditions including, but not limited to, obesity, dyslipidemia and hypercholesterolemia.

The present invention relates to new compositions and methods for using the compositions to modulate thyroid hormone metabolism in extrathyroidal tissues expressing the orphan nuclear receptor CAR and to regulate basal metabolic rate and cholesterol and lipid metabolism in a subject via alteration in the expression and/or activity of the orphan nuclear receptor CAR. Examples of extrathyroidal tissues expressing CAR include, but are not limited to, liver, skeletal muscle, heart, brain, kidney, and intestine.

A global analysis of mouse liver genes differentially expressed in response to CAR activation was performed. Changes in mRNA steady state levels were assessed using AFFYMETRIX® chip technology. AFFYMETRIX® GeneChip murine genome assays (Mu11k set), representing greater than 13,000 full-length genes and EST clusters, were used. The primary sequence sources for this set were combined Unigene and TIGR databases. Selective and potent activation of the CAR nuclear receptor was observed with the CAR agonist 1,4-bis-[2-(3,5-dischloropyridyloxy)] benzene (TCPOBOP). TCPOBOP is a potent and selective activator of mouse CAR and a useful chemical tool for in vivo differential gene expression studies.

In these experiments, three mice were treated with 1 mg/kg TCPOBOP in 5% dimethyl sulfoxide (DMSO) and corn oil, intraperitoneally. Control mice (n—3) received vehicle alone. Each mouse received two injections at 24 hour intervals. Livers were harvested after the second injection. Thus, total treatment time was 28 hours. Total mRNA was isolated from the livers using the Trizol method and probes for hybridization to the Affymetrix chips were prepared from the total mRNA. Results were analyzed by Affymetrix microarray Suite version 4.0 software.

Overall, 264 of more than 13,000 genes displayed a 2-fold or greater change (either in or decrease) in expression in response to TCPOBOP treatment. Two mRNAs, the CYP2B10 mRNA and the CYP3A1 mRNA showed a 40-fold and 20-fold upregulation in expression, respectively, based on the Affymetrix chip data. Elevation of others genes involved in phase, I, II and III metabolism was observed as well as multiple genes involved in thyroid hormone metabolism.

For example, initial Affymetrix data showed that two sulfotransferase mRNAs were altered by TCPOBOP treatment. Since sulfotransferase activity is important in thyroid metabolism, the effect of TCPOBOP on expression of all of the known mouse sulfotransferase genes was examined. The sulfotransferase set examined included N-SULT, SULT-X1, SULT-X2, aryl sulfotransferase, phenyl sulfotransferase, hydroxysteroid sulfotransferase, SULT-B1, and SUTL1E1. Three of these eight sulfotransferase genes were induced by TCPOBOP. Northern blot analysis revealed the N-SULT was increased 11.5-fold and SULT-X1 was increased by 10.0-fold. Using RTQ-PCR, SULT-X2 was shown to increase by 30-fold. Sulfotransferase activity coupled with deiodinase type I activity caused an increased proportion of T4 to be converted to rT3 rather than T3.

Other genes involved in hepatic thyroid hormone conjugation and sport out of the liver were also examined. Conjugation, including glucuronysyl and glutathione transfer, plays an important role in elimination of thyroid hormone in the liver. Using RTQ-PCR, it was found that TCPOBOP caused a 1.6-fold increase in UDP-glucuronysyl transferase 1A1 and increases in several glutathione-S transferase (GST) genes. Namely, 2.7-fold increases were observed in GST theta 1-1 and GST mu1-1, a 4.7-fold increase was observed in GST mu5-5, and a 7.5-fold increase was observed in GST-Y. TCPOBOP also increased the expression of the basolateral transporter, mrp3, which is an efficient carrier of glucuronidated, glutathione-conjugated, and sulfated metabolites (Keppler, D. and Konig, J. Semin. Liver Dis. 2000 20(3):265-72).

Several of the gene changes detected are predicted to lower hepatic T31 levels. For example, Affymnetrix chip data predicted a significant, greater than 2-fold decrease in the gene encoding the Na+/K+ transporting ATPase. This gene is an established T3 target gene and plays a critical role in the effect of T3 on cellular energy utilization. The decrease in the expression of this gene is believed to be an indirect effect of TCPOBOP on T3 levels.

A physiological state that results in an acute lowering of thyroid hormone levels is fasting. Experiments were performed to assess whether the same genes induced by the CAR activator TCPOBOP were also induced by fasting. The fasting study was carried out for 24 hours. During this study, expected declined in total serum T3 and T4 levels were observed.

The expression of several genes examined in the TCPOBOP study was also examined. Of the sulfotransferase genes, SULT-X1, N-SULT, SULT 1E1, SULT 1B1 and aryl sulfotransferase were examined. It was found that sulfotmmsferase mRNAs that were not induced by TCPOBOP (SULT-1E1, SULT-1B1 and aryl sulfotransferase) were also not induced by fasting while the sulfotransferases that were induced by TCPOBOP, N-SULT and SULT-X1, were also induced by fasting. Interestingly, a circadian rhythm was observed in sulfotransferases induced by TCPOBOP and fasting but not in sulfotransferases not induced by TCPOBOP or fasting. Another target gene, UGT-1A1 showed a similar induction pattern after both TCPOBPO treatment and fasting.

Experiments were also conducted to determine directly whether CAR was activated in response to fasting. An established surrogate marker for CAR activation is the expression of CYP2B10 mRNA. The Cyp2b10 gene contains a phenobarbital response element (PBREM) that binds CAR and is activated in in vitro reporter studies by CAR. Expression of CYP2B10 mRNA was examined over a 24 hour time course and was found to increase after 18 hours of fasting. By 24 hours, CAR mRNA was also increased by 1.6 fold.

The sulfotransferase genes regulated by CAR appear to be normally linked to dietary regulation as seen by the fact that the two sulfotransferases regulated by CAR also display a circadian rhythm, being more active at night (when feeding is occurring) than during the day. This type of circadian rhythm is also seen in Cyp7a, a gene involved in cholesterol metabolism. As observed for the sulfotransferases, the circadian rhythm of Cyp7a expression is not disrupted by fasting (Layery et al. Mol. Cell Biol. 1999 19(10):6488-6499). The circadian rhythm of Cyp7a is determined by the circadian regulator DBP (albumin D-site-binding protein), a leucine zipper-type transcription factor (Layery et al. Mol. Cell Biol. 1999 19(10):6488-6499; Wuarin et al, J. Cell Sci. Suppl. 1992 16:123-7). It is possible that SULT-N and SULT-N are coordinately regulated in a circadian fashion by DBP with other metabolically related genes such as Cyp7a. The circadian rhythm of these genes implies that these genes are intimately associated with diet and energy utilization as is the case with Cyp7a.

Thus, data from these experiments are demonstrative of CAR having a role in regulation of thyroid hormone metabolism. This role for CAR is imposed on the normal modes of thyroid hormone regulatory mechanisms and specifically targets extrathyroidal tissues expressing CAR. While liver was the target tissue studied herein, it is expected that other tissues expressing CAR, such as skeletal muscle, heart, brain, intestine and kidney will exhibit similar effects.

Thus, the present invention provides new methods for identifying compositions for modulation of thyroid hormone metabolism based upon the ability of a test agent to modulate CAR expression and/or activity. Test agents that suppress or reduce CAR expression and/or activity, also referred to as antagonists of CAR, are expected to be useful in the treatment of diseases wherein reduced metabolism of thyroid hormone is desired. Accordingly, the present invention also relates to compositions comprising agents that modulate CAR expression and/or activity as well as methods of using these agents to alter thyroid hormone metabolism in a subject.

For purposes of the present invention, by “modulation”, “modulate”, or “modulator” it is meant to regulate, adjust or alter physiological conditions or parameters associated with CAR. Examples of modulation include, but are not limited to, an agent either increasing or decreasing gene expression or activity of the CAR, alterations in timing of expression of this nuclear receptor, increases or decreases in thyroid hormone metabolism, and alterations in basal metabolic rate and/or cholesterol or lipid metabolism. In a preferred embodiment of the present invention, CAR expression and/or activity is suppressed or decreased, thereby resulting in a decease in metabolism of thyroid hormone.

By “alter”, “altering” or “alteration” for purposes of the present invention, it is meant that thyroid hormone levels such as T3 and T4 are increased or decreased upon administration of a modulator of CAR expression and/or activity as compared to levels of these thyroid hormones prior to administration of the modulator.

By “agent” or “test agent” for purposes of the present invention, it is meant to be inclusive of any molecule that increases or decreases CAR expression and/or activity. Most preferred is a molecule that is an antagonist of CAR expression and/or activity. In a preferred embodiment, the agent is a small organic molecule. It is also preferred that the molecule be a ligand for CAR.

Various assays for identifying ligands of CAR can be used.

For example, ligands for use in the compositions of the present invention can be identified routinely through screening of libraries of compounds using assays such as the FRET assay as described in Parks, D. J. 1999. Science 284:1365-1368 and in WO 00/25134. FRET assays comprise the steps of exposing a sample portion comprising the donor located at a first position and the acceptor located at the second position to light at a first wavelength capable of inducing a first electronic transition in the donor, wherein the donor comprises a complex of lanthanide chelate and a lanthanide capable of binding the chelate and wherein the spectral overlap of the donor emission and acceptor absorption is sufficient to enable energy transfer from the donor to the acceptor as measured by a detectable increase in acceptor luminescence. Various coactivators for use in FRET assays have been described. Examples include, but are not limited to, Steroid Receptor Complex (SRC1), CREB binding protein (CBP), and Retinoid Interacting Protein (RIP 140). When a ligand binds to the ligand pocket of the receptor, the coactivator forms a receptor-coactivator complex. The current model on coactivators is that a ligand binds to the ligand binding domain (LBD) causing the activation function 2 (AF-2) to fold into place and trapping the ligand in the pocket. A novel interface (LXXLL motif) is formed by entrapment of the ligand, allowing the coactivator to interact with the AF-2. Thus, AF-2 is important in ligand dependent transactivation. When an inducer or agonist binds, it is transcriptionally active, while the binding of an inhibitor or antagonist interrupts the receptor cofactor interaction.

Cell free binding assays in which CAR, or the ligand binding domain of CAR (alone or present as a fusion protein), can also be performed. In these assays, CAR or the ligand binding domain of CAR is incubated with a test agent that, advantageously, bears a detectable label (e.g. a radioactive or fluorescent label). CAR, or the ligand binding domain thereof, free or bound to the test agent, is then separated from free test agent using any of a variety of techniques (e.g., using gel filtration chromatography (for example, on Sephadex G50 spin columns) or through capture on a hydroxyapatite resin). The amount of test agent bound to CAR or the ligand binding domain thereof, is then determined via detection of the label.

An alternative approach for detecting radiolabeled test agent bound to CAR, or the ligand binding domain thereof, is a scintillation proximity assay (SPA). In this assay, a bead (or other particle) is impregnated with scintillant and coated with a molecule that can capture CAR, or the ligand binding domain thereof (e.g., steptavidin-coated beads can be used to capture biotinylated CAR ligand binding domain). Radioactive counts are detected only when the complex of radiolabeled test agent and the CAR, or ligand binding domain thereof, is captured on the surface of the SPA bead bringing the radioactive label into sufficient proximity to the scintillant to emit a signal. This approach has the advantage of not requiring the separation of free test agent from bound (Nichols et al, Anal. Biochem. 257:112-119 (1998)).

Assays to determine whether a test agent interacts with a CAR ligand binding domain can also be performed via a competition binding assay. In this assay, CAR, or the ligand binding domain thereof, is incubated with a compound known to interact with CAR, which compound, advantageously, bears a detectable label (e.g., a radioactive or fluorescent label). A test agent is added to the reaction and assayed for its ability to compete with the labeled compound for binding to CAR, or ligand binding domain thereof A standard assay format employing a step to separate free known (labeled) compound from bound, or an SPA format, can be used to assess the ability of the test agent to compete.

To determine if a test agent activates CAR, thus increasing thyroid hormone metabolism, the ligand binding domain of CAR is prepared (e.g., expressed) as a fusion protein (e.g., with glutathione-S-transferase (GST), a histidine tag or a maltose binding protein). The fusion protein and coactivator (either or both advantageously labeled with a detectable label, e.g., a radiolabel or fluorescent tag) are incubated in the presence and absence of the test agent and the extent of binding of the coactivator to the fusion protein determined. The induction of interaction in the presence of the test agent is indicative of an activator of CAR.

CAR activation assays in accordance with the invention can be carried out using a full length CAR and a reporter system comprising one or more copies of the DNA binding site recognized by the CAR binding domain. More preferably, however, the activation assays are conducted using established chimeric receptor systems. For example, the ligand binding domain of CAR can be fused to the DNA binding domain of, for example, yeast transcription factor GAL4, or that of the estrogen or glucocorticoid receptor. An expression vector for the chimera (e.g., a GALA-SHP chimera) can be transfected into host cells (e.g., CV-1, HuH7, HepG2 or Caco2 cells) together with a reported construct. The reporter construct can comprise one or more (e.g., 5) copies of the DNA binding site recognized by the binding domain present in the chimera (e.g., the GAL4 DNA binding site) driving expression of a reporter gene (e.g., CAT, SPAP or luciferase). Cells containing the constructs are then treated with either vehicle alone or vehicle containing test agent, and the level of expression of the reporter gene determined. In accordance with this assay, enhancement of expression of the reporter gene in the presence of the test agent indicates that the test agent activates CAR and thus can function as an inhibitor of glucose production.

Another format suitable for identifying ligands of CAR is the yeast two-hybrid assay. This is an established approach to detect protein-protein interactions that is performed in yeast. Protein #1, representing the bait, is expressed in yeast as a chimera with a DNA binding domain (e.g., GALA). Protein #2, representing the predator, is expressed in the same yeast cell as a chimera with a strong transcriptional activation domain. The interaction of bait and predator results in the activation of a reporter gene (e.g., luciferase or beta-galactosidase) or the regulation of a selectable marker (e.g., LEU2 gene). This approach can be used as a screen to detect, for example, ligand-dependent interactions between CAR and other proteins such as coactivator proteins (e.g., SRCI, TIFI, TIF2, ACTR) or fragments thereof (Fields et al., Nature 340:245-2.46 (1989)).

Suitable test agents that can be tested in the above assays include combinatorial libraries, defined chemical entities and compounds, peptide and peptide mimetics, oligonucleotides and natural product libraries, such as display (e.g. phage display libraries) and antibody products.

Typically, organic molecules are screened, preferably small organic molecules that have a molecular weight of from 50 to 2500 daltons. Candidate products are biomolecules including, saccharides, fatty acids, steroids, purines, pyrimidines, derivatives, structural analogs or combinations thereof. Such agents are obtained from a wide variety of sources including libraries of synthetic and natural compounds. Further, known pharmacological agents can be subjected to directed or random chemical modifications, such as acylation, alkylation, esterification, amidification, etc. to produce structural analogs.

Test agents can be used in an initial screen of, for example, 10 agents per reaction, and the agents of these batches that show inhibition or activation tested individually. Test agents may be used at a concentration of from 1 nM to 1000 μM, preferably from 1 μM to 100 μM, more preferably from 1 μM to 10 μM. Preferably, the activity of a test agent is compared to the activity shown by a known activator or inhibitor. A test agent that acts as an inhibitor preferably produces a 50% inhibition of activity of the receptor. Alternatively a test substance that acts as an activator preferably produces 50% of the maximal activity produced using a known activator.

Agents identified as modulators of CAR expression and/or activity can be administered to a subject to alter thyroid hormone metabolism. The ability of an agent to alter thyroid hormone metabolism is expected to be useful in a number of conditions.

For example, anti-obesity therapies and their associated weight loss effects are opposed by homeostatic resistance mechanisms (Spiegelman, B. M. and Flier, J. S. Cell 2001:104(4):531-43). Decreasing thyroid hormone levels is one mechanism that reduces the effectiveness of such therapies. Experiments described herein indicate that CAR contributes to this effect through its effects on genes that regulate thyroid hormone metabolism. Thus, a critical component of the homeostatic resistance to weight loss therapies could be removed by administration of a CAR modulator, and in particular an inhibitor or antagonist of CAR expression and/or activity, in combination with other anti-obesity agents.

Modulators of CAR can also be utilized to effect beneficial changes in cholesterol metabolism. As shown herein CAR agonists regulate aspects of thyroid hormone metabolism, which is turn is known to affect metabolism of cholesterol. Thus, it is believed that modulating CAR will also indirectly affect cholesterol levels. Further, targeting CAR (in contrast to directly targeting thyroid hormone receptor) has the benefit of gaining selective tissue effects in the tissues that express CAR (mainly liver, intestine, and kidney). It is believed that CAR can effect a lowering of cholesterol by increasing sulfation and clearance of bile acids. Accordingly, agents that inhibit or antagonize CAR expression and/or activity are expected to be useful in lowering cholesterol levels and/or increasing clearance of bile acids.

Modulators of CAR can also be utilized to effect beneficial changes in LDL/HDL metabolism. CAR ligands can be used to indirectly modulate specific T3 target genes involved in lowering LDL such as hepatic LDL receptor and lipoprotein lipase. The role of CAR in a normal physiological response to fasting, as demonstrated herein, is indicative of CAR directly modulating a subset of genes involved in cholesterol and lipid metabolism in addition to altering thyroid hormone levels. Accordingly, antagonists of CAR expression and/or activity are also expected to be useful in lowering LDL levels.

Dosing regimes, as well as selection of appropriate routes of administration for compositions comprising an agent of the present invention can be determined routinely by one of skill in the art based upon pharmacological activities of the agent in in vitro and in vivo assays such as described herein. It is preferred that compositions of the present invention comprise an amount of agent which is effective at modulating CAR expression levels or activity so that thyroid hormone metabolism is altered. This effective amount can be determined routinely for each identified agent based upon its activity determined in vitro in screening assays such as described herein and in vivo in animal models. Effective amounts can be confirmed in subjects in need thereof by monitoring the effects of the agent on thyroid hormone levels in the subject. Methods for monitoring thyroid hormone levels in a subject are well known and performed routinely by those skilled in the art.

Agents of the present invention identified as modulators of CAR may be formulated into pharmaceutically acceptable compositions for administration to a subject by any route appropriate for modulation of thyroid hormone metabolism. Suitable pharmaceutical formulations include, but are not limited to, those for oral, rectal, nasal, topical (including buccal and sublingual), vaginal or parenteral (including intramuscular, subcutaneous, intravenous, and directly into the affected tissue) administration or in a form suitable for admistration by inhalation or insufflation. The formulation may, where appropriate be presented in convenient, discrete dosage units and may be prepared by any method well known in the art of pharmacy. All methods include the step of bringing into association the active agent with a liquid or finely divided solid carrier or both and then, if needed, shaping of the product into the desired formulation.

Pharmaceutical formulations suitable for oral administration may be presented in convenient discrete units including, but not limited to, capsules, cachets, or tablets, each containing a predetermined amount of the active agent; as a powder or granules; as a solution, a suspension or as an emulsion. The active agent can also be presented as a bolus, electuary, or paste. Tablets and capsules for oral administration may contain conventional excipients such as binding agents, fillers, lubricants, disintegrants, or wetting agents. The tablets may be coated according to methods well known in the art. Timed-release formulations, which are known in the art, may also be suitable. Oral liquid preparations may be in the form of, for example, aqueous or oily suspensions, solutions, emulsions, syrups or elixirs, or may be presented as a dry product for constitution with water or other suitable vehicles before use. Such liquid preparations may contain conventional additives such as suspending agents, non-aqueous vehicles, including edible oils, or preservatives.

Agents of the present invention identified as modulators of CAR may also be formulated for parenteral administration, such as by injection, for example bolus injection or continuous infusion, and may be presented in unit dose form in ampules, pre-filled syringes, small volume infusion or in multi-dose containers with an added preservative. Pharmaceutically acceptable compositions comprising an active agent for parenteral administration may take the form of suspension, solution or emulsion in oily or aqueous vehicles, and may contain formulatory agents such as suspending, stabilizing, and/or dispersing agents. Alternatively, the active ingredient may be in powder form, obtained by asceptic isolation of sterile solid or by lyophilization from solution, for constitution with a suitable vehicle such as sterile, pyrogen free water, before use.

For topical administration to the epidermis, agents of the present invention identified as modulators of CAR may be formulated as ointments, creams, or lotions, or as a transdermal patch. Ointments and creams, may, for example, be formulated with an aqueous or oily base with the addition of suitable thickening and/or gelling agents. Lotions may be formulated with an aqueous or oily base and will in general also contain one or more emulsifying agents, stabilizing agents, suspending agents, thickening agents, or coloring agents. Formulations suitable for topical administration in the mouth include lozenges comprising an active agent in a flavored base, usually sucrose and acacia or tragacanth; pastilles comprising the active ingredient in an inert base such as gelatin and glycerin or sucrose and acacia; and mouth washes comprising the active ingredient in a suitable liquid carrier. For topical admistration to the eye, the active agent can be made up in solution or suspension in a suitable sterile aqueous or non-aqueous vehicle. Additives such as buffers (e.g. sodium metabisulphite or disodium edeate) and thickening agents such as hypromellose can also be included.

Pharmaceutical formulations suitable for rectal administration wherein the carrier is a solid are preferably presented as unit dose suppositories. Suitable carriers include cocoa butter and other materials commonly used in the art, and the suppositories may be conveniently formed by admixture of the active agent with the softened or melted carrier or carriers followed by chilling and shaping in molds.

Formulations suitable for vaginal administration may be presented as pessaries, tampons, creams, gels, pastes, foams, or sprays containing in addition to the active agent such carriers as are known in the art to be appropriate.

For intra-nasal administration, agents of the present invention identified as modulators of CAR can be used as a liquid spray or dispersible powder or in the form of drops. Drops may be formulated with an aqueous or non-aqueous base also comprising one or more dispersing agents, solubilizing agents, or suspending agents. Liquid sprays are conveniently delivered from pressurized packs.

For administration by inhalation, agents of the present invention identified as modulators of CAR can be delivered by insufflator, nebulizer or a pressurized pack or other convenient means of delivering the aerosol spray. Pressurized packs may comprise a suitable propellant such as dichlorodifluoromethane, trichlorofiluoromethane, dichlorotetrafluoroethane, carbon dioxide or other suitable gas. In the case of a pressurized aerosol the dosage unit may be determined by providing a valve to deliver a metered amount.

Alternatively, for administration by inhalation or insufflation, the active agents of the present invention can take the form of a dry powder composition, for example a powder mix of an agent which modulates CAR and a suitable powder base such as lactose or starch. The powder composition may be presented in unit dosage form in, for example, capsules, cartridges or blister packs of gelatins, from which the powder can be administered with the aid of an inhalator or insufflator.

When desired, any of the above-described formulations may be adapted to provide sustained release of the agents of the present invention identified as modulators of CAR.

The pharmaceutical compositions of the present invention comprising an agent that modulates CAR expression and/or activity can also used in combination with other therapeutic agents.

The amount of an agent of the present invention required for use in treatment will of course vary with the route of administration, the nature of the condition being treated, and the age and condition of the subject being treated. Selection of such an amount, referred to herein as the “therapeutically effective amount or concentration” is ultimately at the discretion of the attending physician. In general, however, suitable doses of pharmaceutical compositions of the present invention providing a therapeutically effective amount of an agent which modulates CAR expression and/or activity will be in the range of from about 0.1 to 300 mg/kg of bodyweight per day, particularly from about 1 to 100 mg/kg of bodyweight per day. An appropriate dosage unit for oral administration generally contains from about 1 to about 250 mg, more preferably 25 to 250 mg of an active agent.

When used in the treatment of obesity, hypercholesterolemia or dyslipidemis, pharmaceutical compositions comprising an antagonist of CAR expression and/or activity can be administered by any of the aforementioned routes, preferably by the oral route or by injection. The daily dosage for a 70 kg mammal will typically be in the range of about 5 mg to 5 grams of active agent of the present invention.

1. A method for identifying test agents that alter thyroid hormone metabolism or cholesterol and lipid metabolism comprising determining a test agent's ability to modulate expression or activity of an orphan nuclear receptor CAR.

2. A composition for alteration of thyroid hormone metabolism or cholesterol and lipid metabolism, said composition comprising an agent which modulates CAR expression or activity in a pharmaceutically acceptable formulation.

3. The composition of claim 2 wherein the agent inhibits or decreases CAR expression or activity.

4. A method for altering thyroid hormone metabolism in a subject comprising administering to a subject in need thereof the composition of claim 2.

5. A method for altering cholesterol and lipid metabolism in a subject comprising administering to a subject in need thereof the composition of claim 3.

6. A method for treating obesity, hypercholesterolemia or dyslipidemia in a subject comprising administering to a subject suffering from obesity, hypercholesterolemia or dyslipidemia the composition of claim 3.

7. The method of claim 6 wherein the subject is suffering from obesity and the composition comprising the agent which inhibits or decreases CAR expression or activity is administered in combination with an anti-obesity agent.
